(19) United States
(12) Patent Application Publication
(10) Pub. No.: US 2025/0179592 A1
(43) Pub. Date: Jun. 5, 2025

(54) METHOD OF DETERMINING PERSONAL EXPOSURE TO A VIRUS

(71) Applicant: Yale University, New Haven, CT (US)

(72) Inventors: Krystal Pollitt, New Haven, CT (US); Jordan Peccia, New Haven, CT (US)

(21) Appl. No.: 18/845,009

(22) PCT Filed: Mar. 8, 2023
(86) PCT No.: PCT/US2023/063975

§ 371 Date: Sep. 9, 2024

Related U.S. Application Data
(60) Provisional application No. 63/318,322, filed on Mar. 9, 2022.

(51) Int. Cl. C12Q 1/70; G01N 1/22

(52) U.S. Cl. CPC C12Q 1/701; G01N 1/2273; C12Q 2600/158; G01N 2001/2276

(57) ABSTRACT

Described herein is a method of determining an exposure to a virus by a subject in a duration of time. The method includes attaching to the subject a sampler for collecting the virus for the duration of time, and detecting a presence or absence of the virus collected by the sampler.

### CROSS-REFERENCE TO RELATED APPLICATIONS

The present application claims priority under 35 U.S.C. § 119 (e) to U.S. Provisional Patent Application No. 63/318,322 filed Mar. 9, 2022, which is incorporated herein by reference in its entirety.

### STATEMENT REGARDING FEDERALLY SPONSORED RESEARCH

This invention was made with government support under 2030545 awarded by National Science Foundation. The government has certain rights in the invention.

### SEQUENCE LISTING

The ASCII text file named “047162-7372WO1 (01942)_Seq Listing.xml” created on Mar. 6, 2023, comprising 6,765 bytes, is hereby incorporated by reference in its entirety.

### BACKGROUND

Inhalation of virus-laden aerosols and contact with respiratory droplets that are expelled from infected individuals (asymptomatic, pre-symptomatic, and symptomatic) during coughing, sneezing, speaking, or breathing are important routes of transmission for many types of viruses, such as airborne respiratory viruses like SARS-CoV-2. Mitigating the spread of such viruses requires layered infection prevention and control strategies, including the availability of clinical testing, use of masks, distancing, hand hygiene, environmental cleaning, and enhanced ventilation. The effectiveness of many of the above infection prevention and control measures can be evaluated using monitors, such as samplers, that measure airborne levels of virus. However, the cost, size, and maintenance requirements of many of the currently available samplers limit their long-term monitoring ability in certain areas, such as hospital wards, nursing homes, schools, and restaurants.

Moreover, the non-portable nature of many active samplers limits their feasibility to be used as devices for evaluating personal exposures to the viruses.

Therefore, there is a need to develop novel methods of detecting environmental viral levels and personal exposures to viruses using smaller and lighter wearable sampler. The present invention addresses this need.

### SUMMARY

In some aspects, the present invention is directed to the following non-limiting embodiments:

In some embodiments, the present invention is directed to a method of determining virus exposure in a subject.

In some embodiments, the method includes: attaching to the subject a sampler for collecting a virus; and detecting whether the virus is collected by the sampler.

In some embodiments, the virus is an airborne virus.

In some embodiments, the sampler includes a sorbent material, and the sampler collects the virus with the sorbent material.

In some embodiments, the sorbent material includes at least one selected from the group consisting of a polystyrene, a polysaccharide, a polyvinyl alcohol, a polyvinylidene fluoride, and a polysiloxane.

In some embodiments, the sorbent material includes polysiloxane, and the polysiloxane includes polydimethylsiloxane (PDMS).

In some embodiments, the sampler further includes a supporting substrate, and the sorbent material is removably mounted to the supporting substrate.

In some embodiments, the sampler further includes a fastener for attaching to the subject.

In some embodiments, the method further includes extracting from the sampler sorbed virus or a fragment thereof.

In some embodiments, the fragment of the collected virus includes a viral nucleic acid.

In some embodiments, the viral nucleic acid includes DNA, RNA, or combinations thereof.

In some embodiments, the extracted virus or fragment thereof is detected by at least one selected from the group consisting of an antibody or nanobody-based detection assay, a polymerase chain reaction (PCR)-based detection assay, and a plaque assay.

In some embodiments, the method further includes quantifying the amount of the collected virus or fragment thereof.

In some embodiments, the method further includes determining the uptake rate of the virus by the sampler.

In some embodiments, the method further includes determining the exposure level to the virus by the subject based on the quantified amount of the virus collected by the sampler and the uptake rate.

In some embodiments, the method further includes determining the virus concentration in an environment in which the subject stayed for a duration of time based on the quantified amount of the virus collected by the sampler, the uptake rate, and the duration of time.

In some embodiments, the virus average diameter ranges from about 20 nm to about 500 nm.

In some embodiments, the virus includes at least one selected from the group consisting of a coronavirus, an influenza virus, a parainfluenza virus, an adenovirus, a respiratory syncytial virus, a human metapneumovirus, a measles moribillivirus and a rhinovirus.

In some embodiments, the coronavirus includes at least one selected from the group consisting of HCoV-229E, HCoV-OC43, HCoV-HKU1, HCoV-NL63, SARS-CoV, MERS-CoV, and SARS-CoV-2.

In some embodiments, the subject is a mammal.

In some embodiments, the subject is a human.

### BRIEF DESCRIPTION OF THE DRAWINGS

For a more complete understanding of the nature and desired objects of the present invention, reference is made to the following detailed description taken in conjunction with the accompanying drawing figures wherein like reference characters denote corresponding parts throughout the several views.

FIG. 1 depicts an experimental setup for testing viral aerosol uptake by PDMS air sampler in a rotating drum in accordance with some embodiments. A 44.5 L aluminum drum was rotated at a constant speed. Filtered air was directly routed into the drum to supply active sampler make-up air or through a pressure gauge into a 6-Jet Collison Nebulizer to generate aerosols. Aerosol was sampled on the opposite side of the drum. Sampling ports included four retractable lines fit with 2.5 cm long PDMS sorbent tubes for passive sampling. Additional ports were used for active air sampling and real-time particle monitoring.

FIGS. 2A-2B depict the results of uptake experiments in accordance with some embodiments. FIG. 2A: PDMS uptake of Φ6 as a function of cumulative virus exposure. The triplicate measurements from the two experimental tests are displayed. The linear regression was fitted using all individual replicates (N=41), and the dash lines indicate the 95% confidence bounds. FIG. 2B: Uptake rates of passive samplers for airborne aerosol species measured by rotating drum in the present study (a non-limiting virus, bacteriophage) and reported in the field studies for various fungal aerosols, trace metals, and particle-phase persistent organic species. The uptake rates were plotted against a variety of affecting factors (aerosol species, sampler type or configuration, seasons, etc.) after being normalized based on the effective collection area of passive samplers. Error bars represent measurement uncertainties.

FIG. 3 depicts the distribution of SARS-CoV-2 RNA concentrations in indoor air based on PDMS Fresh Air Clip passive sampling by sampling location compared to previously reported SARS-CoV-2 RNA concentrations in indoor air using active sampling methods, in accordance with some embodiments. Black circles indicate samples which were deemed positive for SARS-CoV-2 RNA (above the MDL with both replicates positive), grey circles depict samples which were above the MDL but only one replicate was positive, thus the sample was not counted as positive for SARS-CoV-2, and the hollow circle samples reported levels of SARS-CoV-2 RNA which fell below the MDL. The percentages specify the percentage of samples per sampling location which were positive for SARS-CoV-2.

FIGS. 4A-4B depict the representative number (FIG. 4A) and volume (FIG. 4B) size distributions of aerosols in the rotating drum, in accordance with some embodiments. The geometric mean diameters (GMDs) for the particle number and volume distributions were (0.68±0.02) and (1.84±0.25) μm, respectively.

FIGS. 5A-5B depicts the recovery of SARS-CoV-2 RNA (FIG. 5A) and Φ6 RNA (FIG. 5B) from PDMS pads in accordance with some embodiments. Recovery was determined based on measurement of RNA copies extracted from PDMS and directly in aqueous suspensions. The ranges of RNA copies in tested standards covered the expected RNA ranges of collected samples.

FIG. 6 depicts the aerosol concentration decay as a function of time for sizes of 0.3, 0.5, 1, 2.5, and 5 μm, in accordance with some embodiments. Calculated size-fractionated first order decay constants are shown.

FIGS. 7A-7B depict the number of Φ6 RNA copies collected in per unit area of PDMS after exposure in the rotating drum as a function of cumulative exposure to bulk aerosols (FIG. 7A) and cumulative exposure to Φ6 (FIG. 7B) in accordance with some embodiments. All individual replicates instead of the average value for each exposure were used for the regression.

FIGS. 8A-8B depict the relationship between number of extracted Φ6 RNA copies from gelatin filter and the total mass of aerosol collected on gelatin filters, in accordance with some embodiments. Error bars indicate the measurement uncertainties.

FIG. 9 depicts a PDMS passive sampler in accordance with some embodiments. Viral aerosols and droplets containing SARS-CoV-2 are deposited on the PDMS material inside the Fresh Air Clip, worn on a subject's shirt.

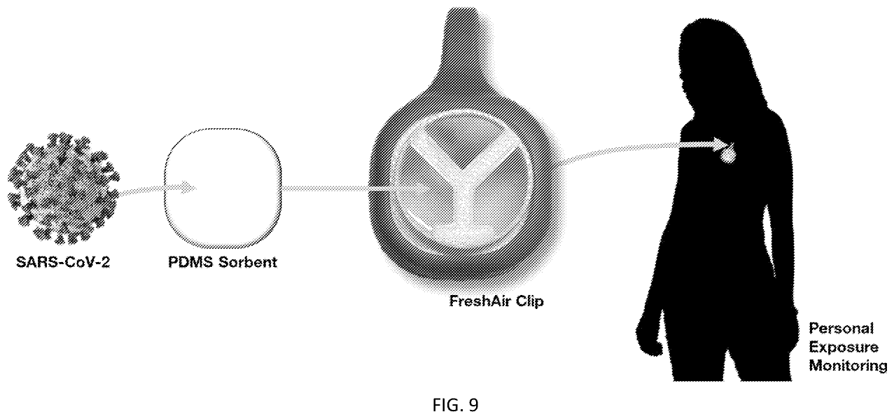

FIG. 10 depicts the distribution of the amount of SARS-CoV-2 RNA copies present on individual PDMS Fresh Air Clips worn by each participant by sampling location in accordance with some embodiments. Black circles indicate samples which were deemed positive for SARS-CoV-2 (above the MDL with both replicates positive), grey circles depict samples which were above the MDL but only one replicate was positive, thus the sample was not counted as positive for SARS-CoV-2, and the hollow circles reported levels of SARS-CoV-2 RNA which fell below the MDL.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10 or less of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

Abbreviations: ABS: acrylonitrile butadiene styrene; MDL: method detection limit; PDMS: polydimethylsiloxane.

### Wearable Pathogen Sampling Device

In some aspects, the present invention is directed to a wearable pathogen monitoring device. In some aspects, the present invention is directed to a method of manufacturing the device. In some aspects, the present invention is directed to a method of extracting a pathogen collected by the device. In some embodiments, the present invention is directed to a method of analyzing a pathogen collected by the device.

Wearable pathogen monitoring devices, associated methods of manufacture, and methods for pathogen analysis are described herein. The device can concentrate airborne pathogens onto a substrate, which can subsequently be analyzed for a broad range of compounds using polymerase chain reaction or other biological assays, mass spectrometry (MS) (with or without chromatography), spectroscopy, nuclear magnetic resonance, electronic detectors, or other analytical platforms. Longitudinal exposure assessment in vulnerable populations can be facilitated by the lightweight, wearable form factor of the device. The low cost of this sampling technique can further enable deployment across large populations, increasing the quantity of environmental data available for evaluating environmental risk factors for disease. The device can include a polydimethylsiloxane (PDMS) sorptive extraction technique to passively concentrate airborne pathogens. A thin PDMS pad can be mounted into a chamber fit into a wearable attachment such as a clip. The wearable device can be worn by an individual for several hours to days depending on ambient levels.

Time-averaged personal exposure concentrations can be evaluated for a broad range of pathogens. The analysis can also include removing background contamination, performing quality control, and associating compounds annotated with uses and potential sources.

### Method of Determining Exposure to Virus

In some aspects, the present invention is directed to a method of determining virus exposure in a subject.

In some embodiments, the method is a method of determining virus exposure in a subject. In some embodiments, the method includes attaching to the subject a sampler for collecting the virus for the duration of time; and detecting whether the virus is collected by the sampler.

In some embodiments, the subject is a mammal, such as a human.

### Probe for Sorbing Virus

In some embodiments, the configuration of the sampler is the same as or similar to those as described elsewhere herein. One of ordinary skill in the art would understand that, although the device as described elsewhere herein is configured for capturing pollutant, such device is able to capture viruses, such as viruses suspended in aerosols.

In some embodiments, the sampler includes a sorbent material, and wherein the sampler collects the virus with the sorbent material.

In some embodiments, the sorbent material is substantially flat. In some embodiments, a height of the sorbent material about 50% or less, such as about 40% or less, about 30% or less, about 20% or less, or about 10% or less of the length and/or width of the sorbent material.

In some embodiments, the sorbent material includes a polystyrene, a polysaccharide, a polyvinyl alcohol, a polyvinylidene fluoride, a polysiloxane, and combinations thereof. In some embodiments, the sorbent material includes polydimethylsiloxane (PDMS).

In some embodiments, the sorbent material is a non-porous material having a smooth surface such that the virus is able to be collected onto the smooth surface. In some embodiments, the sorbent material is a porous material such that the virus is able to be captured by the pores in the material.

In some embodiments, the sampler further includes a supporting substrate. In some embodiments, the sorbent material is removably mounted to the supporting substrate. In some embodiments, the sorbent material is able to be removed from the sampler for viral extraction, and be cleaned for reuse or replaced. In some embodiments, the supporting substrate has one or more openings such that at least a portion of the sorbent material mounted to the supporting substrate is exposed.

In some embodiments, the sorbent and/or the supporting substrate is contained in a housing chamber.

In some embodiments, the sampler further includes an outer cover. In some embodiments, the sampler further includes a fastener for attaching to the subject. Examples of fasteners include magnetic fasteners, loop and hook fasteners, a string loop, a button, and the like. In some embodiments, the fastener is attached to the housing chamber.

### Detection and Quantification of Collected Pathogen

In some embodiments, the method includes performing an extraction of a pathogen, such as but not limited to a virus, or fragment thereof, collected by the sampler. In some embodiments, the fragment of the collected virus comprises viral nucleic acid. In some embodiments, the viral nucleic acid comprises RNA. In some embodiments, the viral nucleic acid comprises DNA.

In some embodiments, the extracted virus or fragment thereof is detected by an antibody-based detection assay, a polymerase chain reaction (PCR)-based detection assay, a plaque assay, or combinations thereof. One of ordinary skill in the art would be able to select proper detection method based on the nature of the virus, the available equipment and material, detection requirements, and the like. For example, when the amount of the virus collected by the sampler is expected to be large and/or there is no quantification requirement or there is no need for accurate quantification, antibody or nanobody-based detection assays (such as ELISA) are sometimes chosen because such assays are able to provide results more quickly. When the amount of the virus collected by the sampler is expected to be small and/or there is a need to provide relatively accurate quantification of the collected virus, PCR-based detection assays such as real-time PCR (RT-PCR) are sometimes chosen as such assays are able to detect low quantity of nucleic acid and are more accurate.

In some embodiments, the method further includes quantifying an amount of the virus collected by the sampler.

### Determining Uptake Rate of Virus by Sampler

In some embodiments, the method further includes determining an uptake rate of the virus by the sampler. Uptake rate means unit volume of air sampled per unit time per unit area of sorbent material in the sampler. For example, the uptake rate can be represented by m³ of air sampled per hour per cm² of sorbent material in the sampler, but the instant specification is not limited thereto.

In some embodiments, determining the uptake rate of the virus by the sampler includes placing the sampler in an environment having a known concentration of the virus. By quantifying the virus collected onto the sorbent material after a predetermined duration, one of ordinary skill in the art is able to calculate the volume of the air corresponding to the amount of the collected virus. Since the sampling time is known and the area of the sorbent material can be determined relatively easy, the uptake rate can hence be determined.

One of ordinary skill in the art would understand that, since the virus being detected by the sampler can be hard to handle in a controlled environment, or harmful or even deadly, a second virus having the same or similar sorbent characteristics to the virus being detected is sometimes used to determine uptake rate instead. In some embodiments, the second virus has similar size, shape, surface components (e.g., lipid envelope vs. glycosylated proteins vs. non-glycosylated proteins), and genomic features (e.g., single-stranded DNA vs. double-stranded DNA vs. single-stranded RNA vs. double-stranded RNA, linear vs. circular vs. segmented, etc.) to the virus the sampler is designed to detect.

In some embodiments, the environment having the known concentration of the virus is produced by preparing a liquid including a known concentration of the test virus (or the second virus), and nebulizing the liquid to form an aerosol containing the virus.

In some embodiments, the uptake rate (R), expressed as m³ of air sampled per hour per cm² of sorbent material in the sampler, is derived as follows:

$$R = \frac{{m}_{NA}}{{\overline{C}}_{NA} \cdot t} = \frac{{m}_{NA}}{{C}_{{NA}_{PM}} {\overline{C}}_{PM} \cdot t} \qquad ( 1 )$$

where m_{NA} (viral nucleic acid copies/cm² of sorbent material) denotes the viral nucleic acid loading on unit area of the sorbent material, and C̅_{NA} (viral nucleic acid copies/m³) is the time-weighted average virus concentration in the drum air over the sampling duration t. The denominator C̅_{NA}·t, as a whole, is a measure of the cumulative exposure to virus during the sampling period, and is calculated by multiplying the virus concentration contained in the test environment (C_{NA_PM}; copies/μg of aerosol) by the cumulative sorbent material exposure to viral aerosols (C̅_{PM}·t; [μg·m⁻³]·hr).

In some embodiments, the virus concentration contained in the test environment C_{NA_PM} is determined from active samples:

$${C}_{NA \_ PM} = \frac{{m}_{NA \_ AS}}{{C}_{PM \_ sampling} {V}_{air}} \qquad ( 2 )$$

where m_{NA_AS} (copies) is the nucleic acid amount measured from, for example, an active sampler, C_{PM_sampling} (μg/m³ of air) is the average mass concentration of suspended aerosol in the test environment during active sampling, and Vₐᵢᵣ (m³) is the air volume passed through the active sampler.

In some embodiments, the cumulative aerosol exposure by the sorbent material of the sampler is calculated as the sum of time-integrated mass concentrations of aerosols with different sizes, with the assumption of a first-order aerosol decay:

$${\overline{C}}_{PM} \cdot t = {\sum }_{i = 1}^{5} {\int }_{{t}_{1}}^{{t}_{2}} {C}_{PM 0 \_ i} {e}^{- {k}_{i} t} dt \qquad ( 3 )$$

where C̅_{PM} (μg/m³) is the time-weighted average mass concentration of aerosols suspended in the drum air during t=t₁˜t₂. C_{PM0_i} (μg/m³) is the initial concentration of aerosol in the i^{th} size bin and kᵢ is its corresponding decay constant, respectively. In some embodiments, the decay constants for aerosols with different sizes is calculated based on particle counter measurements. In some embodiments, the mass concentrations of aerosol are converted from number concentrations assuming spherical aerosols and unity aerosol density (i.e., ρ=1 g/cm³).

### Determination of Environmental Viral Concentration and Personal Viral Exposure

In some embodiments, the method further includes determining the exposure level of the virus in the air (such as the amount of the virus the subject was exposed to) during a duration of time. In some embodiments, the level of exposure to the virus by the subject is determined based on the quantified amount of the virus collected by the sampler and the uptake rate.

In some embodiments, the method further includes determining virus concentration in an environment in which the subject stayed for a duration of time. In some embodiments, the concentration of the virus in the environment is determined based on the quantified amount of the virus collected by the sampler, the uptake rate, and the duration of time.

In some embodiments, the calculated uptake rate (R), the number of viral copies per cm² of the sorbent material (m_{NA}), and the sampling duration (t) are used to estimate the time-weighted average viral aerosol concentration (C̅_{NA}) or the personal exposure level to airborne virus over the assessment period (C̅_{NA}·t) in accordance with Equation (1) as described in the previous section.

### Pathogens Detectable by the Methods of the Disclosure

In some embodiments, the pathogen is a virus. In some embodiments, the virus is airborne. In some embodiments, an average diameter of the virus ranges from about 20 nm to about 500 nm.

In some embodiments, the virus is a coronavirus, an influenza virus, a parainfluenza virus, an adenovirus, a respiratory syncytial virus, a human metapneumovirus, a measles morbillivirus, a rhinovirus., or combinations thereof. In some embodiments, the virus includes a human coronavirus that is capable of infecting a human subject. Examples of human coronaviruses include HCoV-229E, HCoV-OC43, HCoV-HKU1, HCoV-NL63, SARS-CoV, MERS-CoV, and SARS-CoV-2. In some embodiments, the virus includes SARS-CoV-2.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. For example, it should be understood, that modifications in reaction and/or assay conditions, with art-recognized alternatives and using no more than routine experimentation, are within the scope of the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Passive Air Sampler to Assess Personal Exposure to SARS-CoV-2

Example 1 describes the development and application of a polydimethylsiloxane (PDMS)-based passive air sampler to assess personal exposure to SARS-CoV-2 virus.

Exhaled respiratory droplets and aerosols can carry infectious viruses and are an important mode of transmission for COVID-19. Recent studies have been successful in detecting airborne SARS-CoV-2 RNA in indoor settings using active sampling methods. The cost, size, and maintenance of these samplers, however, limit their long-term monitoring ability in high-risk transmission areas. As an alternative, passive samplers can be small, lightweight, inexpensive, and do not require electrical power or maintenance for continual operation. Integration of passive samplers in wearable designs can be used to better understand personal exposure to respiratory virus. The study described in Example 1 evaluated the use of a polydimethylsiloxane (PDMS)-based passive sampler as a non-limiting example to assess personal exposure to aerosol and droplet SARS-CoV-2. The uptake rate of virus-laden aerosol on PDMS was determined in lab-based rotating drum experiments to estimate time-weighted averaged airborne viral concentrations from passive sampler viral loading. The passive sampler was then embedded in a wearable clip design and distributed to community members across Connecticut to surveil personal SARS-CoV-2 exposure. Virus was detected on clips worn by five of the 62 participants (8%) with personal exposure ranging from 4 to 112 copies SARS-CoV-2 RNA/m³, predominantly indoor restaurant settings. Findings demonstrate that PDMS-based passive samplers may serve as a useful exposure assessment tool for airborne viral exposure in real-world high-risk settings and provide avenues for early detection of potential cases and guidance on site-specific infection control protocols that preempt community transmission.

### Example 1-1

COVID-19, caused by Severe Acute Respiratory Syndrome Coronavirus 2 (SARS-CoV-2), was declared a global pandemic by the World Health Organization in March 2020, with over 263.5 million confirmed cases and 5.2 million deaths worldwide, to date (February 2022). Inhalation of virus-laden aerosols and contact with respiratory droplets that are expelled from infected individuals (asymptomatic, pre-symptomatic, and symptomatic) during coughing, sneezing, speaking, or breathing are central routes of transmission for SARS-CoV-2. Mitigating the spread of SARS-CoV-2 and other airborne respiratory viruses requires layered infection prevention and control strategies, including the availability of clinical testing, use of masks, distancing, hand hygiene, environmental cleaning, and enhanced ventilation.

The effectiveness of many of the above infection prevention and control measures can be evaluated using monitors that measure airborne levels of virus. Recent studies have been successful in detecting airborne SARS-CoV-2 RNA in indoor settings using active sampling methods. However, the cost, size, and maintenance of these samplers limit their long-term monitoring ability in high-risk transmission areas, including hospital wards, nursing homes, schools, and restaurants. Moreover, the non-portable nature of many active samplers limits their feasibility to be used as a wearable device for evaluating personal exposures. As an alternative, passive samplers can be small, lightweight, inexpensive, and do not require electrical power or maintenance for continual operation. The broad integration of passive samplers in wearable designs can be used to better understand personal exposure to respiratory virus. While passive sampling is promising from a deployment perspective, it does provide additional challenges including uncertainties in regards to aerosol uptake conditions and higher detection limits compared to active sampling.

The study uses a non-limiting wearable passive air sampler, referred to herein as “Fresh Air Clip,” to monitor personal exposure to airborne SARS-CoV-2. The Fresh Air Clip is a low-cost and lightweight device composed of polydimethylsiloxane (PDMS) which was used to evaluate individual exposure to airborne pathogens that are collected by the polymeric surface. Additional studies have also used PDMS as a model surface for salivary protein adsorption and demonstrated its ability to efficiently capture non-polar compounds, such as lipid enveloped viruses. In the second study, the use of a rotating drum was employed to investigate the uptake rate of virus-laden aerosol on PDMS to estimate time-weighted average airborne viral concentrations from passive sampler viral loading results. Fresh Air Clips then were distributed to community members across Connecticut to surveil personal SARS-CoV-2 exposure. Monitoring airborne SARS-CoV-2 with wearable sampling devices could facilitate risk assessments for virus transmission, providing avenues for early detection of potential cases and guidance on site-specific infection control protocols that preempt community transmission.

### Example 1-2: Material and Methods

### Experimental Uptake Rate Determination

Viral Surrogate Φ6. The bacteriophage Phi6 (Φ6) was used as a BSL-1 surrogate organism to estimate the uptake rate of virus-laden aerosols by PDMS. Φ6 was explored as a surrogate for various enveloped viruses in environmental exposure and persistence studies and was recently utilized as a surrogate for SARS-CoV-2 owing to its similar physiological characteristics to the virus, including diameter ranging from 75 to 100 nm (versus a diameter of 90 to 110 nm for SARS-CoV-2), spherical shape with protruding spike proteins, lipid envelope, and RNA genome.

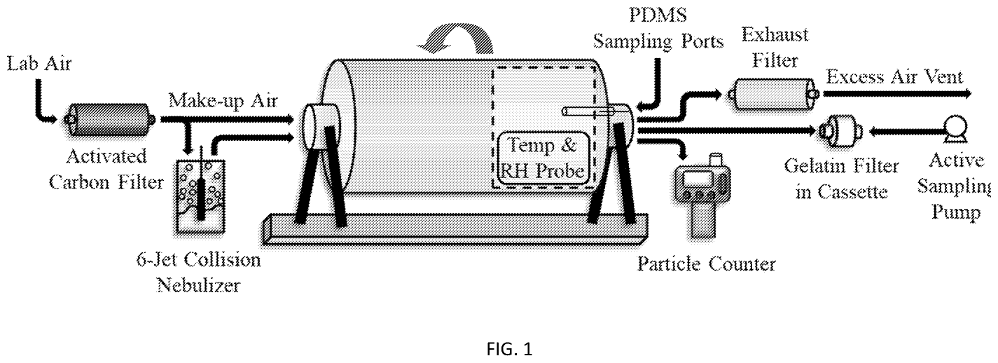

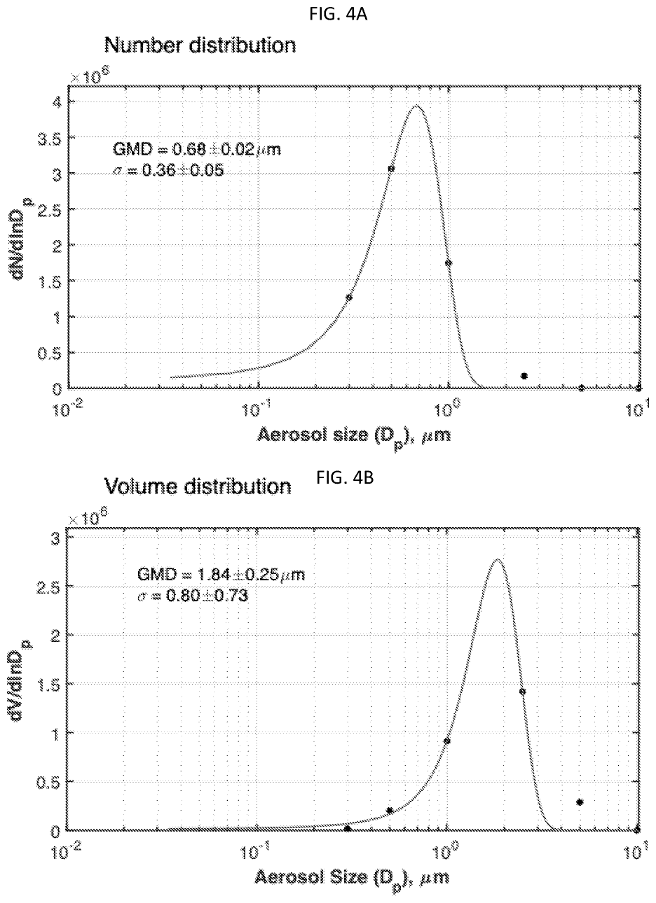

Rotating Drum Configuration, Aerosol Generation and Aerosol Sampling. A custom aluminum drum was constructed to determine the virus-laden aerosol uptake rate by PDMS (FIG. 1). The drum rotated at a constant speed of 2.9 rpm to minimize aerosol loss. Laboratory air was routed through an activated carbon filter to provide purified air to a nebulizer (BGI Inc 6-Jet Collison Nebulizer) and to supply make-up due to losses from active samplers. The drum was maintained at 20° C. throughout tests. Phage lysate in artificial saliva (˜1.9×10⁵ gene copies Φ6/μL; Table 1) was nebulized at 20 psi for 10 seconds to generate polydisperse Φ6-containing aerosols. To better simulate the size of virus-laden aerosols released by infected people through various respiratory activities, CaCl₂) (0.25 M) was added to the nebulizing solution as a coagulant to promote aerosol agglomeration and increased the generation of larger sized aerosol (1.0-5.0 mm). Typical size distributions of the generated aerosols are shown in FIGS. 4A-4B.

TABLE 1
Composition of artificial saliva.

| Chemical species | Amount | | Chemical Species | Amount | |
|---|---|---|---|---|---|
| MgCl₂•6H₂O | 0.04 | g | KSCN | 0.19 | g |
| CaCl₂•2H₂O | 0.15 | g | (NH₂)₂CO | 0.12 | g |
| NaHCO₃ | 0.42 | g | NaCl | 0.88 | g |
| 0.2M KH₂PO₄ | 7.7 | mL | KCl | 1.04 | g |
| 0.2M K₂HPO₄ | 12.3 | mL | Mucin | 3.00 | g |
| NH₄Cl | 0.11 | g | Water (HPLC | 1000 | mL |
| | | | grade) | | |

Passive sampling, active sampling, and real-time size-resolved aerosol measurements were performed three minutes after aerosolization to ensure a homogeneous distribution of aerosol in the drum. For passive sampling, 2.5 cm long PDMS sorbent tubes (effective sampling area of 0.09 cm²; SILASTIC™ Laboratory tubing) were inserted into the drum following aerosolization and were removed after various sampling periods (ranging from 30 minutes to 2 hours). Triplicate PDMS sorbent tube samples were collected for each exposure duration. For active sampling, air was sampled from the drum through a gelatin filter (Sartorius Stedim Biotech) mounted in a filter cassette (SKC Ltd). Active air samples were collected every 30 minutes at a rate of 1.5 L/min for 2 min over the two-hour test periods. Aerosol number concentration was monitored before and after active sampling using a size-resolved (<0.3, 0.3-0.5, 0.5-1.0, 1.0-2.5, 2.5-5.0, and 5.0-10 μm) optical particle counter (MET ONE HHPC-6 Airborne Particle Counter), with the average of two measurements used as the aerosol concentration at that time point. Passive and active air samples collected from uptake experiments were stored in microcentrifuge tubes at −80° C. prior to viral RNA extraction and quantification. Replicate uptake rate experiments were conducted.

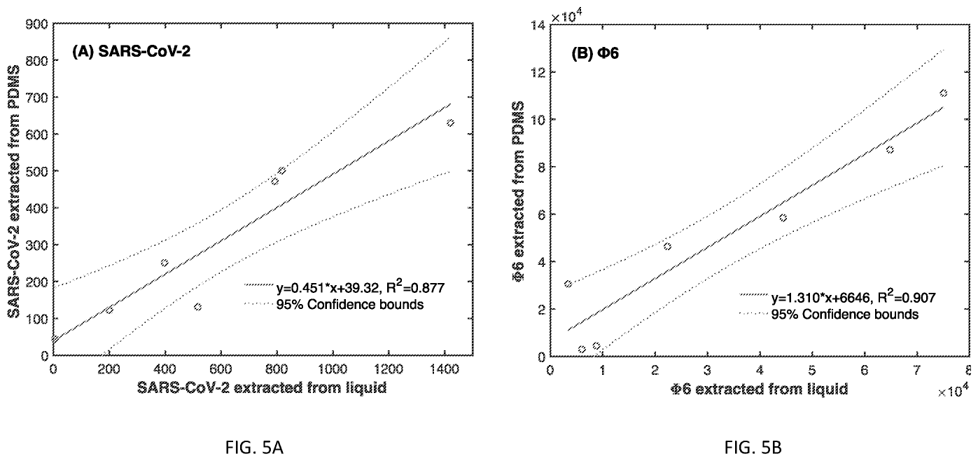

Calculating Uptake Rate of Viral Aerosols on PDMS. To estimate airborne viral time-averaged concentrations using PDMS passive air samplers, the uptake rate of virus-laden aerosols by this sorbent was determined based on active measurements. This uptake rate (R), expressed as m³ of air sampled per hour per cm² of PDMS, was derived as follows:

$$R = \frac{{m}_{RNA}}{{\overline{C}}_{RNA} \cdot t} = \frac{{m}_{RNA}}{{C}_{{RNA}_{PM}} {\overline{C}}_{PM} \cdot t} \qquad ( 1 )$$

where m_{RNA} (RNA copies/cm² of PDMS) denotes the viral RNA loading on unit area of PDMS, which was back-calculated based on the recovered RNA quantities and the virus recovery from PDMS (131%±19% and 45%±8% for Φ6 and SARS-CoV-2, respectively; FIGS. 5A-5B), and C̅_{RNA} (RNA copies/m³) is the time-weighted average virus concentration in the drum air over the sampling duration t (hr). The denominator C̅_{RNA}·t, as a whole, is a measure of the cumulative exposure to virus during the sampling period, and it was calculated by multiplying the Φ6 concentration contained in the aerosols (C_{RNA_PM}; copies/μg of aerosol) by the cumulative PDMS exposure to viral aerosols (C̅_{PM}·t; [μg·m⁻³]·hr).

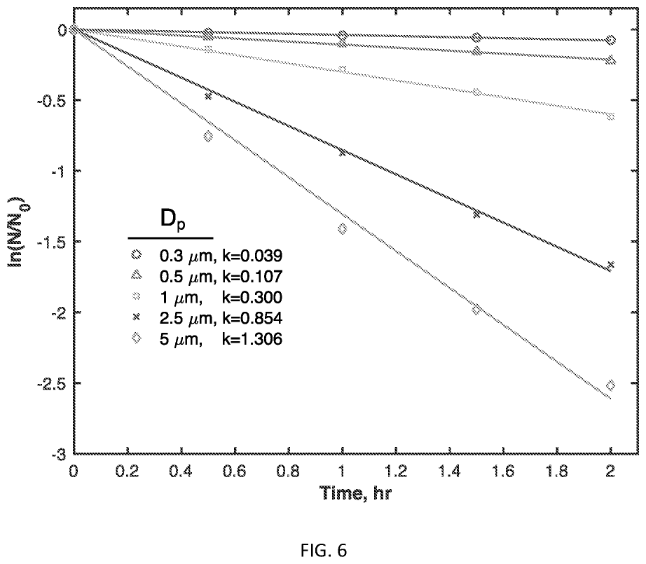

C_{RNA_PM} was determined from active samples:

$${C}_{RNA \_ PM} = \frac{{m}_{RNA \_ filter}}{{C}_{PM \_ sampling} {V}_{air}} \qquad ( 2 )$$

where m_{RNA_filter} (copies) is the RNA amount measured from the gelatin filter, C_{PM_sampling} (μg/m³ of air) is the average mass concentration of suspended aerosol in the drum air during active sampling, and Vₐᵢᵣ (m³) is the air volume passed through the gelatin filter. Assuming first-order aerosol decay in the drum, the cumulative aerosol exposure by PDMS was calculated as the sum of time-integrated mass concentrations of aerosols with different sizes:

$${\overline{C}}_{PM} \cdot t = {\sum }_{i = 1}^{5} {\int }_{{t}_{1}}^{{t}_{2}} {C}_{PM 0 \_ i} {e}^{- {k}_{i} t} dt \qquad ( 3 )$$

where C̅_{PM} (μg/m³) is the time-weighted average mass concentration of aerosols suspended in the drum air during t=t₁˜t₂. C_{PM0_i} (μg/m³) is the initial concentration of aerosol in the i^{th} size bin and kᵢ is its corresponding decay constant, respectively. The decay constants for aerosols with different sizes were calculated based on particle counter measurements (FIG. 6). The mass concentrations of aerosol were converted from number concentrations assuming spherical aerosols and unity aerosol density (i.e., ρ=1 g/cm³). Detailed calculations are described in SI.

The calculated uptake rate (R), the number of viral copies per cm² of PDMS (m_{RNA}), and the Fresh Air Clip sampling duration (t) were then used to estimate the time-weighted average viral aerosol concentration (C̅_{RNA}) or the personal exposure level to airborne virus over the assessment period (C̅_{RNA}·t) in accordance with Equation 1.

### Assessment of Personal Exposure to Airborne SARS-CoV-2 Using the Fresh Air Clip

A PDMS pad (4.10 cm² effective sampling area) was fabricated (Dow Sylgar 184 Silicone Encapsulant Clear Kit) and embedded in a 3D printed acrylonitrile butadiene styrene (ABS) chamber. A perforated cover was also 3D printed from ABS, placed over the PDMS-containing chamber, and mounted in a magnetic clip. This wearable passive air sampler design was referred to as the Fresh Air Clip.

Fresh Air Clips were deployed to individuals across Connecticut, USA between January and May 2021 with participants residing in communities with high COVID-19 transmission rates or working in high-risk indoor occupational environments, such as restaurants offering indoor dining, a homeless shelter, and healthcare facilities. To capture exposure of the breathing zone (i.e., the area near mouth and nose) and allow for sufficient sampling of exposure event opportunities for airborne virus detection, the study participants wore Fresh Air Clips on their shirt collars for five days during their work shifts. Community members living in regions with high COVID-19 transmission wore the Fresh Air Clip during their daily activities (i.e., work-from-home, exercise, shop). Occupational sampling was performed only while study participants were at work. Study participants placed the passive samplers in sealed plastic bags while asleep (community members) or while not at work (occupational). Participants were instructed to wear the Fresh Air Clips during their normal workday or daily activities for five days and completed a Qualtrics survey detailing the dates, duration, and location the Fresh Air Clip were worn as well as their activities during the sampling period. A total of 62 Fresh Air Clips were collected from study participants. The PDMS passive samplers were stored individually at −80° C. after collection. Approval for this study was obtained by the Institutional Review Board at Yale University (HIC #2000026109).

### Virus Quantification

Φ6 RNA was quantified for samples (PDMS sorbent tubes and gelatin filters) collected from rotating drum experiments. SARS-CoV-2 RNA concentrations were determined for the Fresh Air Clips collected from study participants. Viral RNA was extracted from each sample type (Quick-RNA Viral Kit, Zymo Research) and quantified by droplet digital Polymerase Chain Reaction (ddPCR), with corresponding primer/probes (Turgeon et al., Applied and Environmental Microbiology 80 (14) (2014) 4242-4250, Gendron et al., Aerosol Science and Technology 44 (10) (2010) 893-901, and Lee et al., Environmental Science & Technology Letters 3 (5) (2016) 210-215), using the One-Step RT-ddPCR Advanced Kit for Probes (BioRad). Thermocycling was performed according to the manufacturer's recommended protocol with an annealing/extension temperature of 60° C. for Φ6 and 55° C. for SARS-CoV-2 samples. Details can be found in the SI.

### Details of Drum Experiments

### Φ6 Propagation

Pseudomonas syringae pv. phaseolicola strain HB10Y, American Type Culture Collection (ATCC) #21781, was used as the bacterial host to propagate Φ6. A single colony of P. syringae was picked from Luria Broth (LB) agar plates (15.0 g/L agar), two days post streaking, suspended in LB media, and incubated overnight at 25° C. The soft agar overlay plaque assay method was used to propagate working stocks of Φ6, on a lawn of P. syringae, and quantify their infectious concentrations. To harvest phage lysate, soft agar of plates with 20-200 well defined plaques was scraped into a sterile 50 mL conical tube, resuspended in 4 mL of LB media, vortexed, centrifuged at 1200 RPM for 10 minutes and supernatant was passed through a 0.22 μm syringe filter. Stocks of phage lysate containing 10⁶ PFU/μL were used in subsequent rotating drum experimentation.

### Rotating Drum Experiments

The rotating drum is 30.5 cm in outer diameter and 61 cm in length with a total volume of 44.5 L. The drum was attached via belt to an electric motor, rotating at a constant speed of ˜2.9 RPM to maintain suspension of aerosols. Probe and sampling ports were placed on the double sealed ball bearing which comprises the non-rotating part of the drum. The temperature and relative humidity inside the drum were monitored with a Temp/RH probe (Onset HOBO MX1101). Two inlet ports were installed to introduce aerosols and make-up air separately. The nebulizer was supplied with filtered house air (Whatman™ Carbon Cap capsule filter). Multiple sampling ports placed on the other end of the drum were used for insertion of PDMS tube pieces via syringes, filter-based active sampling, monitoring of aerosol size distribution, and exhaust. The exhaust flow was also passed through a HEPA filter to prevent the virus release.

Before each experiment, the drum was cleaned with 90% ethanol and purged with filtered house air until no aerosols were detected. The nebulizing medium, which included phage, artificial saliva, and CaCl₂) was kept on ice and sampled prior to aerosolization to determine the total number of viral genome copies. Test aerosols were generated by a 6-Jet Collison Nebulizer (BGI) at 20 psi and were introduced into the drum for 10 seconds. The exhaust was open during aerosolization to prevent pressure build-up inside the drum. After aerosolization, the drum was sealed and mixed for 3 min to obtain homogeneous aerosol prior to sampling. Multiple 2.5 cm long PDMS tubes (SILASTIC™ Laboratory tubing, 0.2-cm O.D.) were inserted into the drum through the sampling ports and were replaced after different periods of exposure, allowing various exposure durations in different exposure conditions. PDMS samples were collected in triplicate for each exposure. The drum air was withdrawn every 30 min onto a 37 mm gelatin filter in a cassette (SKC Ltd) at a rate of 1.5 L/min for 2 min via an active sampling pump (GilAir Plus). The concentration and size distribution of drum aerosols were monitored before and after each active sampling using an airborne particle counter (Met One HHPC-6). To balance for air loss during sampling, 2 L/min make-up air was supplied into the drum and the excess air was vented.

Two experimental runs were conducted in this study, generating 18 passive PDMS samples (triplicates from 6 exposure conditions) and 4 active filter samples for run #1 and 24 passive samples (triplicates from 8 exposure conditions) and 4 active samples for run #2. All samples were stored immediately after collection at −80° C. prior to RNA extraction and quantification.

### Estimation of Cumulative PDMS Exposure to Viral Aerosols

The viral aerosol concentration in the drum is subject to exponential decay.

$$N = {N}_{0} {e}^{- kt} \qquad ( S1 )$$

where N₀ (#/m³ of air) is the initial aerosol number concentration, N (#/m³ of air) is the aerosol number concentration after time t (hr), and k (hr⁻¹) is the first order aerosol decay constant. The decay constants for aerosols with different sizes were calculated and shown in FIG. 6.

Based on the aerosol decay curve, the cumulative exposure to viral aerosols with a certain aerosol size during t=t₁˜t₂ can be calculated as the integral of aerosol number concentrations versus exposure duration.

$${\int }_{{t}_{1}}^{{t}_{2}} {N}_{0} {e}^{- kt} dt = \frac{{N}_{0}}{- k} {e}^{- kt} {\vert}_{{t}_{1}}^{{t}_{2}} \qquad ( S2 )$$

The aerosol exposure measured by count were further converted to an exposure expressed by aerosol mass assuming spherical particles and unity particle density (i.e., ρ=1 g/cm³). The cumulative exposure to aerosols was calculated as the sum of time-integrated mass concentrations of aerosols with different sizes.

$${\overline{C}}_{PM} t = {\sum }_{i = 1}^{N} M ( {D}_{pi} ) = {\sum }_{i = 1}^{N} ρ \frac{1}{6} π {D}_{pi}^{3} N ( {D}_{pi} ) \qquad ( S3 )$$

where Dₚ is the aerosol diameter, N (Dₚ) and M (Dₚ) are the time integrated number and mass concentrations, respectively, for aerosols with size of Dₚ. C̅_{PM} (μg/m³ of air) is the time-weighted average mass concentration of aerosols suspended in the drum air.

The RNA concentration contained in the aerosol (C_{RNA_PM}, in unit of copies/μg) was then determined from active samplers.

$${C}_{RNA \_ PM} = \frac{{m}_{RNA \_ filter}}{{C}_{PM \_ sampling} {V}_{air}} \qquad ( S4 )$$

where m_{RNA_filter} (copies) is the RNA amount measured from the gelatin filter, C_{PM_sampling} (μg/m³ of air) is the average mass concentration of suspended aerosol in the drum air during active sampling, and Vₐᵢᵣ (m³) is the air volume passed through the gelatin filter.

The uptake rate of viruses by PDMS can be expressed as

$$R = \frac{{m}_{RNA}}{{\overline{C}}_{RNA} t} = \frac{{m}_{RNA}}{{C}_{RNA \_ PM} {\overline{C}}_{PM} t} \qquad ( S5 )$$

where m_{RNA} (copies/cm² of PDMS) is the amount of RNA accumulated on unit area of PDMS, which was adjusted for recovery of virus from PDMS. C̅_{RNA} (copies/m³) is the time-weighted average virus concentration in the drum air, and t (hr) is the exposure duration. Assuming that the virus concentrations in aerosols with various sizes are identical and remain constant during the whole experiment period, the viral RNA in the drum air follows the same decay pattern as the aerosols. Therefore, the cumulative exposure to virus, i.e., C̅_{RNA}t, is equal to C_{RNA_PM} times the cumulative exposure to aerosols C̅_{PM}t.

### Viral Quantification and Recovery

### PDMS Pad Synthesis

Using the 184 Silicone Encapsulant Clear 0.5 kg kit (Dow Sylgard™), PDMS was syringed onto a substrate and desiccated overnight to remove bubbles, creating a smooth surface for sorption. Elevated open covers were added to the PDMS pads to prevent direct contact with other surfaces but allow for aerosol diffusion and droplets to land on the PDMS surface. PDMS pads were then secured in Fresh Air Clips for personal sampling.

### Quantification of Φ6 by RT-ddPCR

Φ6 RNA was quantified in the artificial saliva used for aerosol generation in the nebulizer as well as passive air samplers (PDMS sorbent tubes) and active air samples (gelatin filters). RNA extraction used the Quick-RNA Viral Kit (Zymo Research) and eluted into 15 μL of RNase free water. RNA extraction of the Φ6 nebulizing solution was completed by adding 10 μL of sample into the lysis step. Gelatin filters were dissolved in RNase free water (100 μL) and added to the lysis buffer while PDMS sorbent tubes were placed directly into lysis buffer. The extraction protocol was completed according to the manufacturer's instructions. Total viral RNA was quantified using droplet digital Polymerase Chain Reaction (ddPCR), with the One-Step RT-ddPCR Advanced Kit for Probes (BioRad) and the manufacturer's recommended thermocycling protocol with an annealing/extension temperature set to 60° C. Samples were run in duplicates with 5 μL of RNA template per sample replicate. A previously developed primer probe set was used for Φ6, the sequences are as follows: forward primer sequence 5′-TGGCGGCGGTCAAGAGC-3′ (SEQ ID NO:1), reverse primer sequence 5′-GGATGATTCTCCAGAAGCTGCTG-3′ (SEQ ID NO:2), and probe 5′-FAM-CGGTCGTCG/ZEN/CAGGTCTGACACTCGC-IABkFQ-3′ (FAM-SEQ ID NO:3-ZEN-SEQ ID NO: 4-IABkFQ).

### Quantification of SARS-CoV-2 by RT-ddPCR

SARS-CoV-2 RNA concentrations were evaluated for PDMS (4.10 cm²) contained in Fresh Air Clips. PDMS pads were removed from the substrate with forceps, sectioned into strips, directly placed into lysis buffer tubes and extracted using the Quick-RNA Viral Kit (Zymo Research) with an elution volume of 15 μL. The One-Step RT-ddPCR Advanced Kit for Probes (BioRad) and its protocol was used for ddPCR analysis with 5 μL of RNA template per sample, and an annealing/extension temperature of 55° C. The N1 assay of the 2019-nCoV CDC Kit (Integrated DNA Technologies) was used for quantification of SARS-CoV-2 from field samples; sequences as follows: forward primer sequence 5′-GACCCCAAAATCAGCGAAAT-3′ (SEQ ID NO: 5), reverse primer sequence 5′-TCTGGTTACTGCCAGTTGAATCTG-3′ (SEQ ID NO:6), and probe 5′-FAM-ACCCCGCAT/ZEN/TACGTTTGGTGGACC-3IABKFQ-3′ (FAM-SEQ ID NO: 7-ZEN-SEQ ID NO:8-3IABKFQ). Sample extracts were run in duplicates and with negative and positive controls of nuclease free water and SARS-CoV-2 standards (BioRad), respectively.

To confirm the SARS-CoV-2 positivity of Fresh Air Clip samples, two criteria were applied: 1) both replicates of RNA extracts per sample were required to be positive and 2) copies of SARS-CoV-2 viral RNA were above the method detection limit (MDL) of 6 copies/clip following ddPCR analysis.

Viral Recovery from PDMS

The recovery of SARS-CoV-2

from the PDMS pad was determined. Wastewater previously detected to contain SARS-CoV-2 virus was diluted, loaded onto PDMS pads at seven different concentrations (6 to 1420 RNA copies) in duplicate and dried at room temperature for 30 minutes in a biosafety cabinet. RNA was then extracted from the PDMS pads and liquid samples containing equivalent amounts of virus using the Quick-RNA Viral Kit (Zymo Research) and quantified via ddPCR. The recovery for SARS-CoV-2 RNA from the PDMS pads was determined to be 45%±8% SE (95% CI: 30 to 60%) (FIGS. 5A-5B). This recovery experiment was replicated using Φ6, the surrogate for SARS-CoV-2 used in rotating drum experiments. Phage was diluted and the decreasing concentrations of Φ6 were loaded onto PDMS pads. Liquid dilutions of Φ6 as well as the PDMS pads loaded with corresponding dilutions of Φ6 were extracted and quantified. Extraction of Φ6 from PDMS was near unity with average recovery of 131%±19% SE, and a 95% confidence interval for the slope of 80 to 180%. Additionally, experimentation to determine the potential decay in virus recovery over time (the five-day sampling period of Fresh Air Clips) was conducted. SARS-CoV-2 and Φ6 were independently loaded onto PDMS pads and dried for 30 minutes. Viral RNA was extracted from the PDMS pads on days 1, 3, and 5 following viral loading onto the pads and quantified using ddPCR. These results yielded no observable decrease in RNA concentration for SARS-CoV-2 or Φ6 recovery from the PDMS pads over the five-day period at 50% RH.
SARS-CoV-2 ddPCR MDL Determination

Our analysis to estimate the MDL for SARS-CoV-2 using ddPCR was conducted to provide a framework for understanding noise versus signal when small quantities of amplification appear in a sample. A set of 64 blanks were analyzed to estimate a method detection limit for SARS-CoV-2 quantification via ddPCR. The blanks analyzed included nuclease free water (Zymo Research and Invitrogen-Thermo Fisher Scientific) and blank PDMS pads. Method blanks were non-zero 20% of the time. The estimated MDL, determined to be 6 copies/extraction, was determined by adding three times the standard deviation of the method blanks to their mean concentration.

### Example 1-3: Results and Discussion

### Uptake of Virus-Laden Aerosols by PDMS

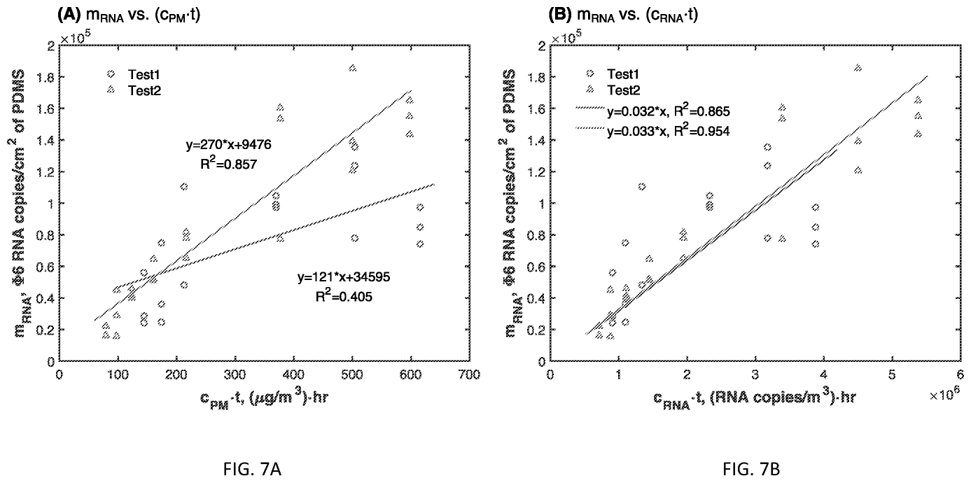

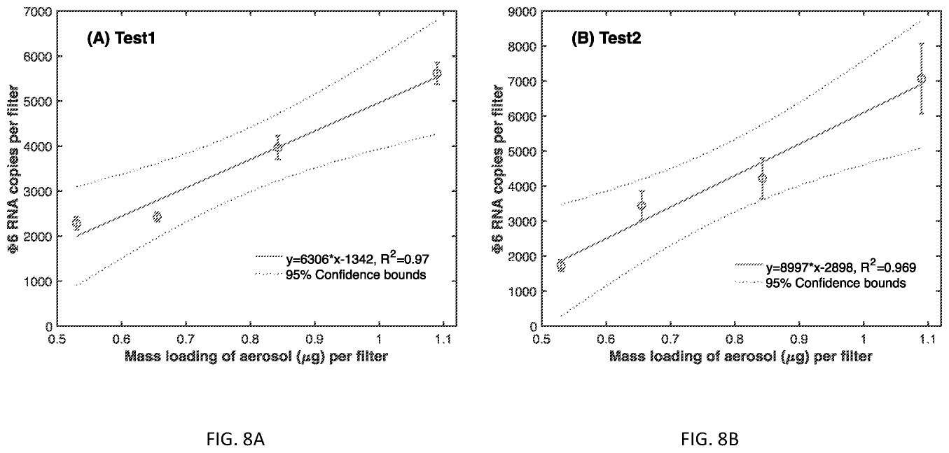

The kinetic uptake of virus-laden aerosols by PDMS was evaluated for cumulative exposure to bulk aerosol ranging from 80 to 600 (μg/m³)·hr (FIG. 7A-7B) with a viral load of 6.3 to 9.0×10³ RNA copies per μg of aerosol (FIGS. 8A-8B). A positive linear relationship was found between the RNA copies sampled by the PDMS and the cumulative Φ6 exposure (FIG. 7B), suggesting the PDMS had not approached its equilibrium uptake capacity during the exposure period. The high aerosol exposure of 600 (μg/m³)·hr (FIG. 7A), therefore, can be used to estimate the time over which linear uptake was expected based on the ambient aerosol levels in different environments. Within the linear uptake regime, the average viral concentration in ambient air over the sampling period can be quantified given the passive sampler's uptake rate.

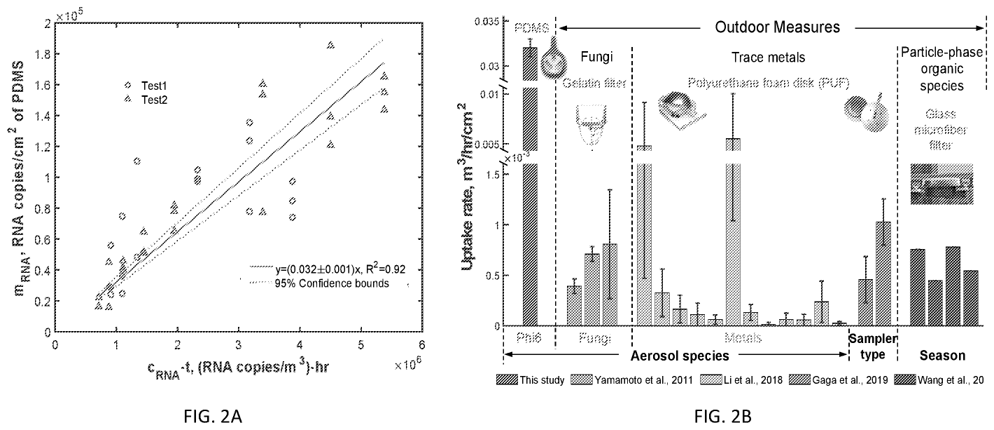

The uptake rates determined from the two experimental tests were found to be similar (p=0.54 for a regression comparison t-test). By combining all the individual observations, the average uptake rate of Φ6 by unit area of PDMS was determined to be 0.032±0.001 m³/hr/cm² (R²=0.92, N=41; FIG. 5A). This experimentally derived uptake rate is higher than the rates reported for other types of absorbents (0.0007 to 0.004 m³/hr/cm²) which were derived based on outdoor measurements of airborne fungi, trace metals, and persistent organic species in aerosols utilizing different passive sampler configurations and collection media (FIG. 2B). The variability in the uptake rates may be due to changes in aerosol composition, aerosol size distribution, sampler types, and environmental conditions (e.g., wind speed). The sheltered design of passive air samplers used in previous published studies served to minimize variable air flow over the sorbent material by controlling the boundary layer of air above the sampling surface. While this design limited variability in the uptake rate of airborne contaminants, the rate of uptake was also reduced. To enhance aerosol deposition for the wearable passive air sampler, the second study used an open-face design that allowed for increased air flow over the PDMS pad. The hydrophobic and porous properties of PDMS likely also enhanced uptake of virus laden aerosol.

### Assessment of Personal Exposure to Airborne SARS-CoV-2 Using the Fresh Air Clip

Sixty-two Fresh Air Clips were returned and assessed: 47 from occupational environments and 15 from community members (Table 2). The uneven distribution of total samplers analyzed per category was due to difficulties of public reliance, particularly in wearing the Fresh Air Clip for 5 days, completing the associated survey, and returning the used clip. While the Fresh Air Clip itself is easily deployable, the sampling duration of 5 days in addition to the necessity of an extended time commitment filling out the survey made it challenging for participants, particularly essential workers in high stress environments, to reliably complete the sampling process.

TABLE 2
Location-based sampling of distributed PDMS Fresh Air Clip passive
samplers, including relevant SARS-CoV-2 infectious rates.

| | | | | | Averaged Estimated |
|---|---|---|---|---|---|
| | | | | | SARS-CoV-2 Daily |
| Exposure | | | | | Case Rate |
| Assessment | Samplers | Samplers | Sampling | Mask | (cases/100,000 |
| Location | Distributed | Returned | Months | Mandate | people) |
| Restaurants | 47 | 19 | March-May | No (while | 27.2 |
| | | | | patrons were | |
| | | | | eating) | |
| Healthcare | 46 | 17 | January- | Yes | 39.5 |
| Facilities | | | April | | |
| Community | 24 | 15 | March-May | Varied | 26.2 |
| Homeless | 26 | 11 | March-May | Yes | 19.0 |
| Shelter | | | | | |

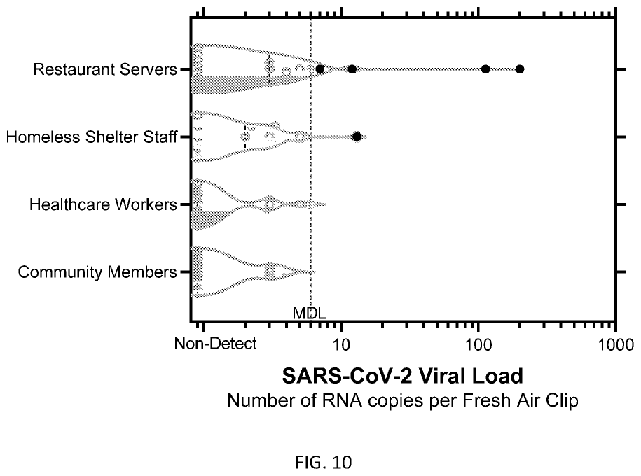

The second study were able to reliably detect samples positive for SARS-CoV-2 with ≥6 copies of viral RNA per sampler extraction, based on analysis of method blanks (detailed in the SI). Eight percent of Fresh Air Clips were positive for SARS-CoV-2 viral RNA, with values ranging from 7 to 200 copies per clip (FIG. 10). This represents the total SARS-CoV-2 viral RNA detected on the passive sampler using ddPCR methods; infectious viral concentrations were not assessed. Of the positive Fresh Air Clips, four were worn by restaurant servers and one by homeless shelter staff. Notably, two positive samples collected in restaurants with indoor dining were found to have high viral load when compared to the other samples (>100 copies per clip), suggestive of close contact with one or more infected individuals. Sampling was conducted when case rates in the communities studied ranged from ˜4 to 102 estimated daily COVID-19 cases per 100,000 people. All locations were under mask mandates during sampling; however, restaurant patrons are not required to wear masks while seated, potentially accounting for the more frequent and higher SARS-CoV-2 values observed in restaurants. Similarly, the lack of SARS-CoV-2 detection in healthcare facilities is fairly expected, as hospitals have strict personal protective equipment (PPE) requirements, cleaning protocols, and high ventilation rates which are associated with decreased transmission.

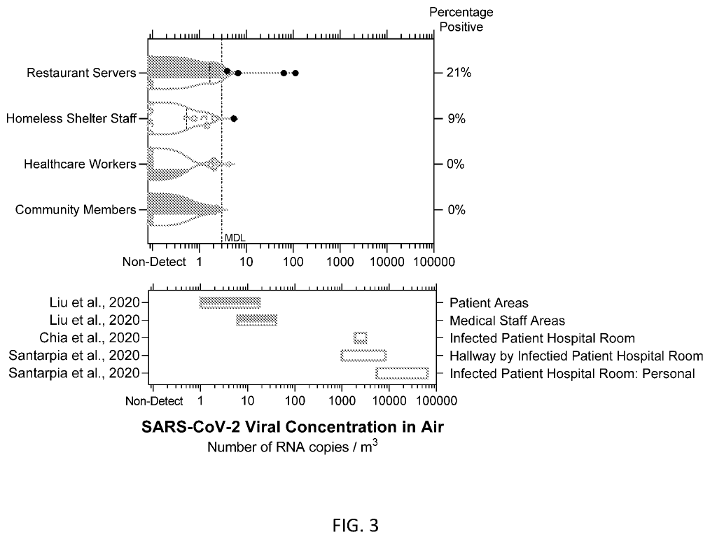

Viral load measurements on positive samplers were converted to the cumulative exposure ([copies/m³]·hr) of corresponding participants to SARS-CoV-2 during deployment (Equation 1), based on the vial uptake rate and the sampler collection area; the results are shown in Table 3. Ambient SARS-CoV-2 concentrations in indoor settings were further determined based on the passive sampling duration of each participant (FIG. 3). Positive samples estimate a range of 4 to 112 copies SARS-CoV-2 RNA copies/m³ (Table 3). The ambient viral levels in this study are comparable to those determined with active gelatin filter sampling in a medical staff area but lower than the levels observed in hospital rooms of infected patients.

TABLE 3
Details of participants' personal exposure to airborne
SARS-CoV-2 are shown. Results are limited to samples found
to be positive for SARS-CoV-2. Exposures are expressed as
viral load, cumulative exposure, and airborne concentrations.

| | | | | Airborne |
|---|---|---|---|---|
| | | | Cumulative | Virus |
| Participants' | SARS-CoV-2 | Assess- | Exposure to | concentra- |
| Exposure | Viral Load | ment | Virus | tion |
| Assessment | (RNA copies/ | Duration | ([RNA copies | (RNA copies |
| Location | clip) | (hr) | m⁻³] · hr) | m⁻³) |
| Restaurant | 7 | 30 | 117 | 4 |
| Restaurant | 12 | 30 | 201 | 7 |
| Restaurant | 113 | 30 | 1891 | 63 |
| Restaurant | 200 | 30 | 3347 | 112 |
| Homeless | 13 | 40 | 218 | 5 |
| Shelter | | | | |

Detection of SARS-CoV-2 using Fresh Air Clips demonstrates that exposure to airborne or droplet virus can be detected using passive sampling methods. The collection of 14 copies of SARS-CoV-2 viral RNA was necessary on a Fresh Air Clip to identify a positive sampler. This is ˜21 times lower than the estimated inhalation dose for SARS-CoV-2, thus the Fresh Air Clip can detect exposure events at sub-infectious doses. While the size of the study population limited comparison between microenvironments, it can be concluded that PDMS passive samplers can serve as a semi-quantitative screening tool for assessing personal exposure to viral aerosols. Scaling the deployment of Fresh Air Clips can facilitate the identification of high-risk areas for indoor SARS-CoV-2 exposure. More broadly, this PDMS passive air sampling tool can be used to create public health situational awareness for the presence of other biological threats to the health of the public.

### Enumerated Embodiments

In some aspects, the present invention is directed to the following non-limiting embodiments:

Embodiment 1: A method of determining virus exposure in a subject, the method comprising:
attaching to the subject a sampler for collecting a virus; and
detecting whether the virus is collected by the sampler.

Embodiment 2: The method of Embodiment 1, wherein the virus is an airborne virus.

Embodiment 3: The method of any one of Embodiments 1-2, wherein the sampler comprises a sorbent material, and wherein the sampler collects the virus with the sorbent material.

Embodiment 4: The method of Embodiment 3, wherein the sorbent material comprises at least one selected from the group consisting of a polystyrene, a polysaccharide, a polyvinyl alcohol, a polyvinylidene fluoride, and a polysiloxane.

Embodiment 5: The method of Embodiment 4, wherein the sorbent material comprises polysiloxane, and wherein the polysiloxane comprises polydimethylsiloxane (PDMS).

Embodiment 6: The method of any one of Embodiments 1-5, wherein the sampler further comprises a supporting substrate, and wherein the sorbent material is removably mounted to the supporting substrate.

Embodiment 7: The method of any one of Embodiments 3-6, wherein the sampler further comprises a fastener for attaching to the subject.

Embodiment 8: The method of any one of Embodiments 1-7, further comprising extracting from the sampler sorbed virus or a fragment thereof.

Embodiment 9: The method of Embodiment 8, wherein the fragment of the collected virus comprises viral nucleic acid.

Embodiment 10: The method of Embodiment 9, wherein the viral nucleic acid comprises DNA, RNA, or combinations thereof.

Embodiment 11: The method of Embodiment 10, wherein the extracted virus or fragment thereof is detected by at least one selected from the group consisting of an antibody or nanobody-based detection assay, a polymerase chain reaction (PCR)-based detection assay, and a plaque assay.

Embodiment 12: The method of any one of Embodiments 10-11, further comprising quantifying the amount of the collected virus or fragment thereof.

Embodiment 13: The method of Embodiment 12, further comprising determining the uptake rate of the virus by the sampler.

Embodiment 14: The method of Embodiment 13, further comprising determining the exposure level to the virus by the subject based on the quantified amount of the virus collected by the sampler and the uptake rate.

Embodiment 15: The method of Embodiment 14, further comprising determining the virus concentration in an environment in which the subject stayed for a duration of time based on the quantified amount of the virus collected by the sampler, the uptake rate, and the duration of time.

Embodiment 16: The method of any one of Embodiments 1-15, wherein the virus average diameter ranges from about 20 nm to about 500 nm.

Embodiment 17: The method of any one of Embodiments 1-16, wherein the virus comprises at least one selected from the group consisting of a coronavirus, an influenza virus, a parainfluenza virus, an adenovirus, a respiratory syncytial virus, a human metapneumovirus, a measles moribillivirus and a rhinovirus.

Embodiment 18: The method of Embodiment 17, wherein the coronavirus comprises at least one selected from the group consisting of HCoV-229E, HCoV-OC43, HCoV-HKU1, HCoV-NL63, SARS-COV, MERS-COV, and SARS-CoV-2.

Embodiment 19: The method of any one of Embodiments 1-18, wherein the subject is a mammal.

Embodiment 20: The method of any one of Embodiments 1-19, wherein the subject is a human.

### EQUIVALENTS

Although non-limiting embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of determining virus exposure in a subject, the method comprising:
attaching to the subject a sampler for collecting an airborne virus;
wherein the sampler comprises a sorbent material,
wherein the sorbent material comprises at least one selected from the group consisting of a polystyrene, a polysaccharide, a polyvinyl alcohol, a polyvinylidene fluoride, and a polysiloxane, and
wherein the sampler collects the virus with the sorbent material; and
detecting whether the virus is collected by the sampler.

2. The method of claim 1, wherein the sorbent material comprises polysiloxane, and wherein the polysiloxane comprises polydimethylsiloxane (PDMS).

3. The method of claim 1, wherein the sampler further comprises a supporting substrate, and wherein the sorbent material is removably mounted to the supporting substrate.

4. The method of claim 1, wherein the sampler further comprises a fastener for attaching to the subject.

5. The method of claim 1, further comprising extracting from the sampler sorbed virus or a fragment thereof.

6. The method of claim 5, wherein the fragment of the collected virus comprises viral nucleic acid.

7. The method of claim 6, wherein the viral nucleic acid comprises DNA, RNA, or combinations thereof.

8. The method of claim 7, wherein the extracted virus or fragment thereof is detected by at least one selected from the group consisting of an antibody or nanobody-based detection assay, a polymerase chain reaction (PCR)-based detection assay, and a plaque assay.

9. The method of claim 7, further comprising quantifying the amount of the collected virus or fragment thereof.

10. The method of claim 9, further comprising determining the uptake rate of the virus by the sampler.

11. The method of claim 10, further comprising determining the exposure level to the virus by the subject based on the quantified amount of the virus collected by the sampler and the uptake rate.

12. The method of claim 11, further comprising determining the virus concentration in an environment in which the subject stayed for a duration of time based on the quantified amount of the virus collected by the sampler, the uptake rate, and the duration of time.

13. The method of claim 1, wherein the virus average diameter ranges from about 20 nm to about 500 nm.

14. The method of claim 1, wherein the virus comprises at least one selected from the group consisting of a coronavirus, an influenza virus, a parainfluenza virus, an adenovirus, a respiratory syncytial virus, a human metapneumovirus, a measles moribillivirus and a rhinovirus.

15. The method of claim 14, wherein the coronavirus comprises at least one selected from the group consisting of HCoV-229E, HCoV-OC43, HCoV-HKU1, HCoV-NL63, SARS-CoV, MERS-CoV, and SARS-CoV-2.

16. The method of claim 1, wherein the subject is a mammal.

17. The method of claim 1, wherein the subject is a human.


SEQUENCE LISTING (8 sequences)

SEQ ID NO: 1 - DNA, 17 bp, synthetic construct
  source 1..17: mol_type = other DNA
tggcggcggtcaagagc

SEQ ID NO: 2 - DNA, 23 bp, synthetic construct
  source 1..23: mol_type = other DNA
ggatgattctccagaagctgctg

SEQ ID NO: 3 - skipped

SEQ ID NO: 4 - DNA, 16 bp, synthetic construct
  source 1..16: mol_type = other DNA
caggtctgacactcgc

SEQ ID NO: 5 - DNA, 20 bp, synthetic construct
  source 1..20: mol_type = other DNA
gaccccaaaatcagcgaaat

SEQ ID NO: 6 - DNA, 24 bp, synthetic construct
  source 1..24: mol_type = other DNA
tctggttactgccagttgaatctg

SEQ ID NO: 7 - skipped

SEQ ID NO: 8 - DNA, 15 bp, synthetic construct
  source 1..15: mol_type = other DNA
tacgtttggtggacc